(19) United States
(12) Patent Application Publication
(10) Pub. No.: US 2023/0372664 A1
(43) Pub. Date: Nov. 23, 2023

(54) CATHETER SET FOR FORMING DIFFERENT CATHETER ARRANGEMENTS FOR PERIPHERAL REGIONAL ANAESTHESIA

(71) Applicant: B. Braun Melsungen AG, Melsungen (DE)

(72) Inventors: Iris Blum, Fuldabrueck (DE); André Weiss, Guxhagen (DE); Nicky Bode, Schimberg Nieste (DE); Jacqueline Felmeden, Melsungen (DE)

(21) Appl. No.: 18/197,261

(22) Filed: May 15, 2023

(30) Foreign Application Priority Data
May 23, 2022 (DE) 10 2022 205 129.7

(51) Int. Cl. A61M 25/00; A61M 19/00

(52) U.S. Cl. CPC A61M 25/0009; A61M 19/00; A61M 2025/0166

(57) ABSTRACT

A catheter set for forming catheter arrangements includes a capillary with a capillary tube and a capillary attachment joined to the capillary tube, a cannula with a cannula tube insertable into the capillary tube, a cannula attachment joined to the cannula tube and connectable to the capillary attachment, a catheter hose advanceable through the capillary tube, a catheter coupling for fastening to the catheter hose, and a fluid admission line with a hose and at least one fluid connector joined to the hose and which is connectable to the capillary attachment. A first catheter arrangement, which has the catheter hose and catheter coupling fastened to the catheter hose, is configured for dispensing anaesthetic via the catheter hose. A second catheter arrangement, which has the capillary and the fluid admission line, is connected to the capillary attachment by the fluid connector and configured for dispensing anaesthetic via the capillary tube.

### CROSS-REFERENCE TO RELATED APPLICATION(S)

This application claims priority under 35 U.S.C. § 119 to German Application No. 10 2022 205 129.7, filed May 23, 2022, the content of which is incorporated by reference herein in its entirety.

### FIELD

The present disclosure relates to a catheter set for forming different catheter arrangements for peripheral regional anaesthesia.

### BACKGROUND

In peripheral regional anaesthesia, the conduction of pain impulses along an individual nerve is blocked in a targeted manner. This is also referred to as nerve blockade or nerve block. The blockade is effected by administering an anaesthetic in direct proximity to the nerve in question.

Catheter arrangements for peripheral regional anaesthesia have been known for some time and are fitted on the patient with the aid of catheter sets. Different procedures for fitting them are known. These different procedures, also referred to as catheter placements, are used according to the particular situation or depending on the preference of the anaesthetist. Known catheter placements are, for example, the “Braunuele technique”, the “catheter-through-needle technique” and the “catheter-over-needle” technique.

Different catheter sets are known for different catheter placements. The known catheter sets are each composed of a number of components, for example a cannula, a capillary, a fluid admission line, etc. For the different catheter placements mentioned, the applicant offers catheter sets under the registered trademarks CONTIPLEX® D, CONTIPLEX® S, CONTIPLEX® TUOHY AND CONTIPLEX® C. In terms of their physical and functional features, the components of the known catheter sets are specifically tailored to the particular catheter placement. Each of the catheter sets is configured for a defined catheter placement and therefore for forming a specific catheter arrangement.

### SUMMARY

The object of the present disclosure is to make available a catheter set of the aforementioned type which affords advantages over the prior art.

The catheter set according to the disclosure has a capillary, a cannula, a catheter hose, a catheter coupling and a fluid admission line. The aforementioned components of the catheter set can be used to selectively form a first catheter arrangement and a second catheter arrangement. The first catheter arrangement is assigned to a first catheter placement. The second catheter arrangement is assigned to a different, second catheter placement. In this way, the catheter set according to the disclosure permits particularly versatile and simplified use. Using the catheter set according to the disclosure, the anaesthetist is able to decide in a short time, according to the situation and/or according to his or her preference, which catheter placement is to be used. The catheter set according to the disclosure also permits simplified production, logistics and storage. To permit two different catheter placements, it has hitherto been necessary for two different catheter sets to be produced, commissioned and kept in stock by the end user. With the catheter set according to the disclosure, matters are simplified in this respect and costs savings are made. The capillary has a capillary tube and a capillary attachment. The capillary attachment is joined to a proximal end of the capillary tube. Joining methods suitable for this purpose are known to a person skilled in the art. The cannula has a cannula tube and a cannula attachment. The cannula attachment is joined to a proximal end of the cannula tube. Joining methods suitable for this purpose are known to a person skilled in the art. In one embodiment of the catheter set, the cannula tube, in a delivery state, is pushed through the capillary attachment into the capillary tube, and the cannula attachment and the capillary attachment are connected releasably to each other. This, for example, by means of a form-fit and/or force-fit connection. The capillary attachment and the cannula attachment are adapted to each other in this respect. The cannula serves in particular for puncturing the patient. The puncture is made in a state with the cannula joined together with the capillary, such that the cannula tube, together with the capillary tube supported on the cannula tube, is advanced into the body tissue of the patient. In a further embodiment, the cannula, in the delivery state, is spatially separate from the capillary. In this case, the cannula tube is able to be pushed through the capillary attachment into the capillary tube. In this case, the canula attachment and the capillary attachment are releasably connectable to each other. The capillary tube and the cannula tube are adapted to each other in dimensions. In particular, an external diameter of the cannula tube is smaller than an internal diameter of the capillary tube. Preferably, an axial length of the canula tube is longer than an axial length of the capillary tube. The catheter hose is able to advance in the distal direction through the capillary tube. For this purpose, a distal hose end is introduced through the capillary attachment into the capillary tube and advanced distally along the capillary tube. The catheter hose and the capillary tube are adapted to each other in dimensions. In particular, an external diameter of the catheter hose is smaller than an internal diameter of the capillary tube. The catheter coupling is configured for fastening to a proximal end of the catheter hose. Catheter couplings are known in principle to a person skilled in the art and serve for fluidically connecting a catheter hose to further fluid-carrying components. A catheter coupling suitable for this purpose is sold by the applicant under the registered trademark PERIFIX®. The fluid admission line has a hose and at least one fluid connector. The at least one fluid connector is joined at the distal end of the hose and is releasably connectable to the capillary attachment. The fluid admission line can also be designated as an extension line. Preferably, the fluid admission line has a further fluid connector, which is joined to a proximal end of the hose.

The first catheter arrangement has the catheter hose and the catheter coupling fastened at the proximal end of the catheter hose. Preferably, the first catheter arrangement is composed of the catheter hose and of the catheter coupling fastened to the latter. Using the first catheter arrangement according to the disclosure, the anaesthetic is dispensed via the catheter hose. The second catheter arrangement has the capillary and the fluid admission line. The latter is connected to the capillary attachment by means of the fluid connector. Preferably, the second catheter arrangement is composed of the capillary and the fluid admission line. Using the second catheter arrangement, the anaesthetic is dispensed via the capillary tube. Admission to the capillary tube is by means of the fluid admission line. To form the first catheter arrangement, the components of the catheter set can be used as follows. In a first step, the canula along with the capillary is pushed into the body tissue. In a further step, the cannula is removed. For this purpose, the cannula attachment is released from the capillary attachment, and the cannula tube located in the capillary tube is pulled out in the proximal direction. In a further step, the catheter hose is fitted. To do this, its distal end is introduced through the capillary attachment into the capillary tube and is advanced distally. In a further step, the capillary is removed. To do this, the capillary tube along with the capillary attachment is pulled proximally away from the catheter hose. In a further step, the catheter coupling is fastened to the proximal end of the catheter hose. To form the second catheter arrangement, the components of the catheter set can be used as follows. In a first step, the canula along with the capillary is pushed into the body tissue of the patient. In a further step, the canula is removed. In this connection, reference is made to what has already been said. In a further step, the fluid admission line is mounted on the capillary. To do this, the fluid connector is connected to the capillary attachment.

In one embodiment of the disclosure, the catheter set has a fixing component. The fixing component is fastenable to the capillary attachment and is configured for axially fixing the catheter hose inserted into the capillary tube. In this embodiment of the disclosure, the catheter set is configured for selectively forming an (additional) third catheter arrangement. The third catheter arrangement has the capillary, the catheter hose and the fixing component. Moreover, the third catheter arrangement has the catheter coupling fastened to the proximal end of the catheter hose. Preferably, the third catheter arrangement is composed of the capillary, the catheter hose, the fixing component and the catheter coupling. In the third catheter arrangement, the fixing component is fastened to the capillary attachment, and the catheter hose for dispensing the anaesthetic is advanced in the distal direction through the capillary tube and is axially fixed relative to the capillary by means of the fixing component. The third catheter arrangement makes it possible to adapt an axial protrusion of the distal end of the catheter hose past the distal end of the capillary tube. For the purpose of adapting the axial protrusion, the catheter hose can to a greater or lesser extent be axially advanced or retracted relative to the capillary. After the protrusion has been adapted, the relative position of the catheter hose with respect to the capillary is fixed by means of the fixing component. In one embodiment, the fixing component is fastened to a proximal end of the capillary attachment. In a further embodiment, the fixing component is at least partially inserted into a receiving recess of the capillary attachment. For fixing, the fixing component interacts with the catheter hose by force-fit and/or form-fit engagement. To do this, the fixing component can have a latching, clamping, eccentric or other fixing mechanism. To form the third catheter arrangement, the components of the catheter set can be used as follows. In a first step, the capillary supported on the cannula is fitted. To do this, the body tissue of the patient is punctured in the region of the nerve that is to be blocked. The cannula tube is advanced together with the capillary tube distally into the body tissue. In a further step, the cannula is removed. What has been said in this connection concerning the first and/or second catheter arrangement applies mutatis mutandis to the third catheter arrangement. In a further step, the fixing component is fitted on the capillary. To do his, the fixing component is fastened to the capillary attachment. The fixing component and the capillary attachment are accordingly adapted to each other. In a further step, the catheter hose is fitted. To do this, the distal end of the latter is introduced through the fixing component and the capillary attachment into the capillary tube and advanced distally along the capillary tube. In a further step, the catheter hose is positioned relative to the capillary. To do this, its distal end is advanced and/or retracted as far as a desired position with respect to the distal end of the capillary tube. In a further step, the catheter hose is fixed. To do this, the fixing component is actuated and the catheter hose is axially fastened relative to the capillary under the action of the fixing component. In a further step, the catheter coupling is fastened to the proximal end of the catheter hose.

In a further embodiment of the disclosure, the capillary tube is designed to be flexible. The flexible properties of the capillary tube are advantageous in particular as regards the second catheter arrangement. In the second catheter arrangement, the anaesthetic is dispensed via the capillary tube. Accordingly, the capillary tube remains in place for the duration of the, in particular continuous, peripheral regional anaesthesia. The flexible design of the capillary tube in particular permits improved comfort for the patient.

In a further embodiment of the disclosure, the capillary tube is produced from polyurethane. The inventors have found that polyurethane affords particular advantages for the production of the capillary tube.

In a further embodiment of the disclosure, the catheter hose has an extractable mandrel. The mandrel facilitates the insertion and advance of the catheter hose. The mandrel can be extracted in the proximal direction from the catheter hose. This embodiment is particularly advantageous when the catheter hose has a comparatively small diameter and is accordingly pliable. The mandrel provides mechanical stabilization of the catheter hose.

In a further embodiment of the disclosure, a plurality of sensors configured for ultrasound-based locating are present and are mounted on different components of the catheter set. Sensors for ultrasound-based locating of medical invasive components are known to a person skilled in the art. In this embodiment, different components of the catheter set, for example the capillary, the cannula and/or the catheter hose, are equipped with such a sensor. The corresponding component is thus “visible” by ultrasound. This permits ultrasound-based locating and/or navigating of the component in question when applied to the patient.

In a further embodiment of the disclosure, a first sensor is integrated in the distal end of the capillary tube. Alternatively or in addition, a second sensor is integrated in the distal end of the cannula tube. Alternatively or in addition, a third sensor is integrated in the distal end of the catheter hose. If the catheter hose has an extractable mandrel, the latter can be provided with the third sensor or with a further, fourth sensor. By equipping the respective component with one of said sensors, position monitoring and/or navigation can be performed based on ultrasound.

In a further embodiment of the disclosure, the fixing component has an actuation element and a dimensionally pliable clamping element, wherein the clamping element, in the third catheter arrangement, is received in a receiving recess of the capillary attachment and has a lumen which is oriented coaxially to the capillary tube and through which the catheter hose extends. The actuation element is mounted on the capillary attachment in such a way as to be movable relative to the capillary attachment. Moreover, it is operatively connected to the clamping element in a force-transmitting and/or movement-transmitting manner. The clamping element is elastically deformable by means of a movement of the actuation element. The elastic deformation takes place between a clamping configuration and an enabling configuration. In the clamping configuration, the clamping element is elastically deformed in such a way that the lumen is radially narrowed and an axial mobility of the catheter hose in the lumen is fixed by frictional engagement. In the enabling configuration, the clamping element is not elastically deformed or is less elastically deformed, such that the radial narrowing of the lumen and thus the frictional engagement are cancelled and the axial mobility of the catheter hose is enabled. The physical and functional features associated with this design of the fixing component are particularly advantageous.

The disclosure moreover relates to a method for forming a first catheter arrangement. The first catheter arrangement is configured for use in, particularly continuous, peripheral regional anaesthesia. The first catheter arrangement is assigned to a first catheter placement. The method for forming the first catheter arrangement has the following steps: providing a catheter set according to the above description; removing the cannula, wherein the cannula attachment is released from the capillary attachment, and the cannula tube advanced in the capillary tube is extracted in the proximal direction from the capillary tube; fitting the catheter hose, wherein the distal end of the latter is inserted into the capillary attachment and advanced in the distal direction along the capillary tube; removing the capillary, wherein the capillary tube is withdrawn in the proximal direction from the catheter hose; fastening the catheter coupling to the proximal end of the catheter hose.

The disclosure moreover relates to a method for forming a second catheter arrangement. The second catheter arrangement is configured for use in, particularly continuous, peripheral regional anaesthesia. The second catheter arrangement is assigned to a second catheter placement. The method for forming the second catheter arrangement has the steps of: providing a catheter set according to the above description; removing the cannula, wherein the cannula attachment is released from the capillary attachment, and the cannula tube advanced in the capillary tube is extracted in the proximal direction from the capillary tube; fitting the fluid admission line, wherein the fluid connector of the latter is connected to the capillary attachment. To connect the fluid connector to the capillary attachment, the fluid connector can be screwed onto the capillary attachment, plugged together with it or in some other way releasably connected to it by force-fit and/or form-fit engagement.

The disclosure moreover relates to a method for forming a third catheter arrangement. The third catheter arrangement is also configured for use in, particularly continuous, peripheral regional anaesthesia. The third catheter arrangement is assigned to a third catheter placement. The method for forming the third catheter arrangement has the steps of: providing a catheter set according to the above description; removing the cannula, wherein the cannula attachment is released from the capillary attachment, and the cannula tube advanced in the capillary tube is extracted in the proximal direction from the capillary tube; fitting the fixing component to the capillary, wherein the fixing component is fastened to the capillary attachment; fitting the catheter hose, wherein the distal end of the latter is inserted into the capillary attachment and is advanced in the distal direction along the capillary tube; positioning the catheter hose, wherein the distal end of the latter is advanced as far as a desired position with respect to the distal end of the capillary tube; fixing the catheter hose, wherein the fixing component is actuated and the catheter hose is axially fixed relative to the capillary under the action of the fixing component; fastening the catheter coupling to the proximal end of the catheter hose. The fixing component is fastened to the capillary attachment by force-fit and/or form-fit engagement. If the fixing component has an actuation element and a clamping element, the actuation element is preferably screwed onto the capillary attachment. The clamping element is preferably inserted into a/the receiving recess of the capillary attachment. To fit the catheter hose, the distal end of the latter is inserted through the fixing component and the capillary attachment into the capillary tube. The catheter hose is positioned by being advanced and/or retracted until a desired axial protrusion of its distal end beyond the distal end of the capillary tube is obtained. If the fixing component has an actuation element and a clamping element according to the above-described embodiment of the catheter set, the clamping element is transferred from the enabling configuration to the clamping configuration in order to fix the catheter hose. This is done by actuating the actuation element.

### BRIEF DESCRIPTION OF THE DRAWING FIGURES

Further advantages and features of the disclosure will become clear from the following description of exemplary embodiments shown in the drawings, of which:

FIG. 1 shows a simplified schematic view of an embodiment of a catheter set according to the disclosure with a capillary, a cannula, a catheter hose, a catheter coupling, a fluid admission line and a fixing component;

FIG. 2 shows a detailed schematic view of the capillary of the catheter set according to FIG. 1;

FIG. 3 shows a detailed schematic view of the cannula of the catheter set according to FIG. 1;

FIG. 4 shows a simplified schematic view of a first catheter arrangement, wherein the first catheter arrangement is formed using the catheter set according to FIG. 1;

FIG. 5 shows a simplified schematic view of a second catheter arrangement formed using the catheter set according to FIG. 1;

FIG. 6 shows a simplified schematic view of a third catheter arrangement formed using the catheter set according to FIG. 1;

FIG. 7 shows the third catheter arrangement according to FIG. 6 in an alternative schematic view;

FIG. 8 shows a partially cutaway longitudinal sectional view, along a section A-A, of the third catheter arrangement according to FIG. 7;

FIG. 9 shows a schematic block diagram of an embodiment of a method according to the disclosure for forming the first catheter arrangement according to FIG. 4 using the catheter set according to FIG. 1;

FIG. 10 shows a schematic block diagram of an embodiment of a method according to the disclosure for forming the second catheter arrangement according to FIG. 5 using the catheter set according to FIG. 1; and

FIG. 11 shows a schematic block diagram of an embodiment of a method according to the disclosure for forming the third catheter arrangement according to FIGS. 6 to 8 using the catheter set according to FIG. 1.

### DETAILED DESCRIPTION

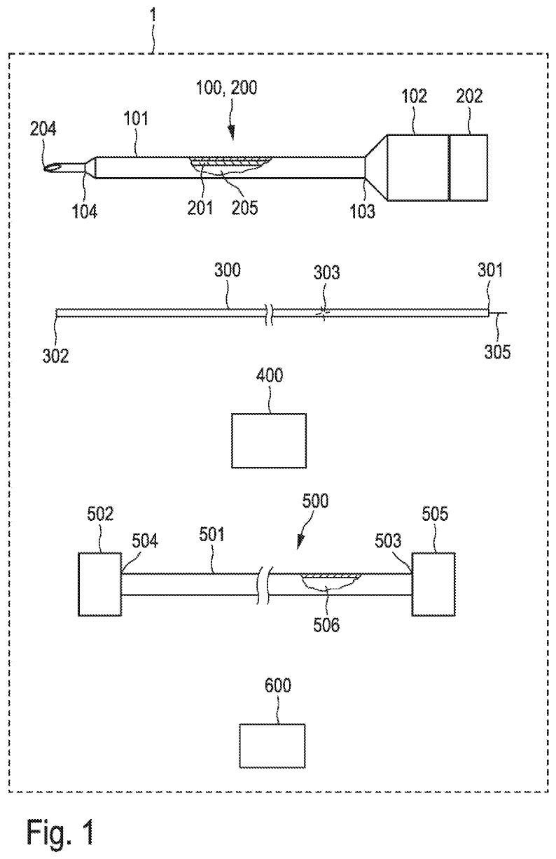

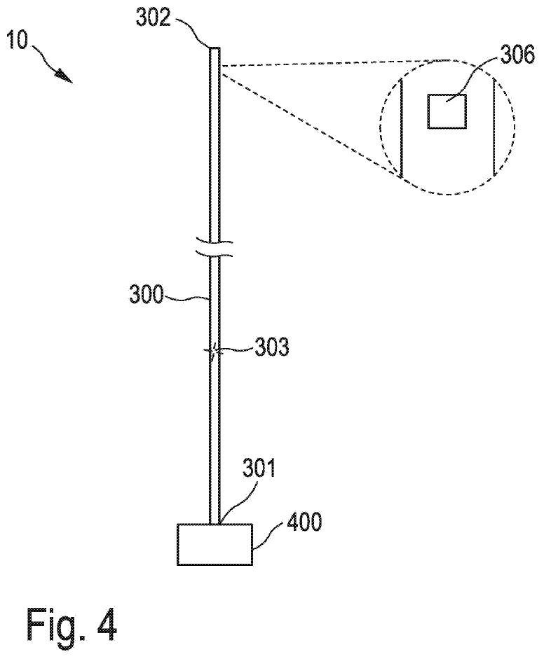

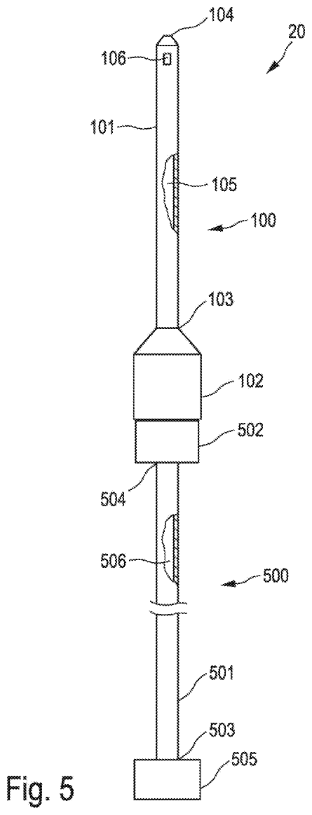

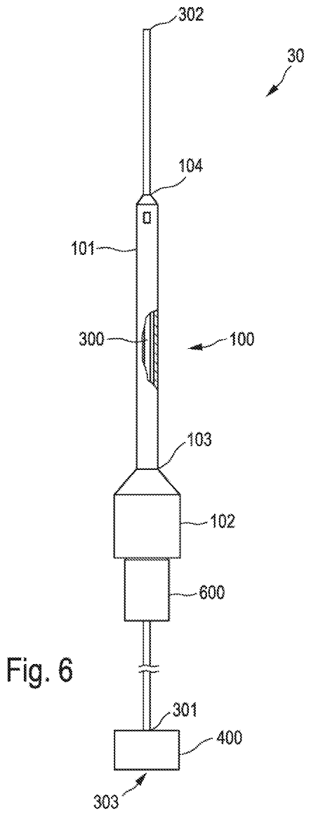

According to FIG. 1, a catheter set 1 for use in peripheral regional anaesthesia is provided. The catheter set 1 is configured for selectively forming different catheter arrangements 10, 20, 30. Specifically, the catheter set 1 is configured for forming a first catheter arrangement 10 (FIG. 4), a second catheter arrangement 20 (FIG. 5) and a third catheter arrangement 30 (FIGS. 6 to 8). To form the different catheter arrangements 10, 20, 30, the components (described in detail below) of the catheter set 1 are combined with one another in different ways.

The catheter set 1 has a capillary 100, a cannula 200, a catheter hose 300, a catheter coupling 400 and a fluid admission line 500.

In the embodiment shown, the catheter set 1 moreover has a fixing component 600. The fixing component 600 is optional. In an embodiment not shown separately in the figures, the catheter set does not have a fixing component.

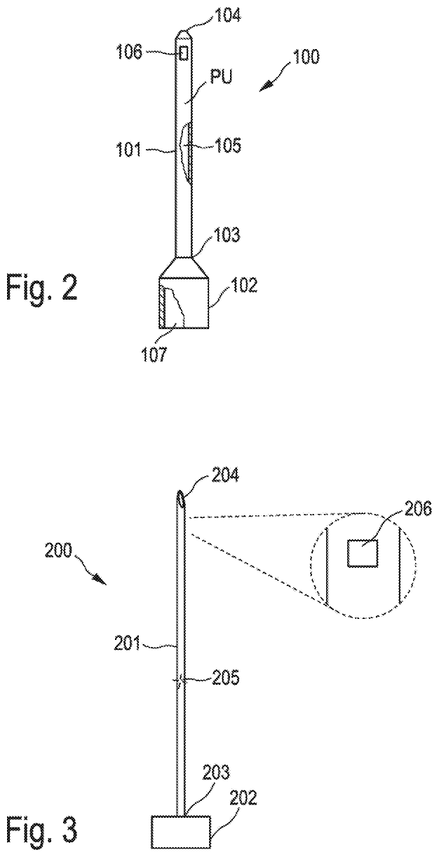

The capillary 100 has a capillary tube 101 and a capillary attachment 102 (see FIG. 2). The capillary attachment 102 is joined to a proximal end 103 of the capillary tube 101. The capillary tube 101 is elongate between the proximal end 103 and a distal end 104. The capillary 100, specifically the capillary tube 101, has a lumen 105. The lumen 105 is continuous from proximal end to distal end. The capillary 100 can also be designated as a short catheter.

In the embodiment shown, the capillary tube 101 is designed to be flexible. The capillary tube 101 is in the present case produced from a flexible plastic material. Specifically, the capillary tube 101 is produced from polyurethane PU.

The cannula 200 has a cannula tube 201 and a cannula attachment 202. The cannula attachment 202 is joined to a proximal end 203 of the cannula tube 201. The cannula tube 201 is elongate between the proximal end 203 and a distal end 204. The distal end 204 has a cannula opening (not shown in detail). The cannula 200, specifically the cannula tube 201, has a lumen 205. The lumen 205 is continuous from proximal end to distal end. The lumen 205 opens at one end into the cannula opening (not shown in detail). The cannula tube 201 is insertable into the capillary tube 101. Moreover, the cannula attachment 202 and the capillary attachment 102 are releasably connectable to each other. FIG. 1 shows an inserted and connected state. To insert the cannula tube 201, its distal end 204 is inserted distally through the capillary attachment 102 into the capillary tube 101, specifically the lumen 105 thereof. In the inserted state, the distal end 204 of the cannula tube 201 protrudes beyond the distal end 104 of the capillary tube 101 (see FIG. 1). The capillary tube 101 and the cannula tube 201 are adapted in dimensions to each other. In particular, an internal diameter of the capillary tube 101 is greater than an external diameter of the cannula tube 201. The internal diameter and the external diameter are without reference signs. Moreover, an axial length of the cannular tube 201 is greater than an axial length of the capillary tube 101. The two axial lengths are likewise without reference signs. The releasable fastening between the capillary attachment 102 and the cannula attachment 202 is by form-fit and/or force-fit engagement. The capillary attachment 102 and the cannula attachment 202 are configured for releasable fastening. The releasable fastening is, for example, a screw, plug and/or clamp connection. Accordingly, the capillary attachment 102 and the cannula attachment 202 are designed at least partially complementing each other.

The catheter hose 300 is elongate between a proximal end 301 and a distal end 302. It will be understood that the catheter hose 300 has a lumen. The lumen is labelled with the reference sign 303 and is continuous from proximal end to distal end. The catheter hose 300 can be advanced in the distal direction through the capillary tube 101. For this purpose, the catheter hose 300 and the capillary 100 are adapted in dimensions to each other. In particular, an external diameter of the catheter hose 300 is smaller than an internal diameter of the capillary tube 101. Said diameters are again without reference signs.

In the embodiment shown, the catheter hose 300 has an extractable mandrel 305. In the configuration shown in FIG. 1, the mandrel 305 protrudes from the proximal end 301 of the catheter hose 300. The mandrel 305 is elongate within the lumen 303. The mandrel 305 counteracts undesired kinking of the catheter hose 300. This facilitates the insertion and advance of the catheter hose 300. The mandrel 305 mechanically stabilizes the catheter hose 300. In an embodiment not shown in the figures, the catheter hose does not have a mandrel.

The catheter coupling 400 is configured for fastening to the proximal end 301 of the catheter hose 300. The nature of the fastening is different in different embodiments. In one embodiment, the catheter coupling 400 is plugged together with the proximal end 301. Alternatively or in addition, clamping or other suitable fixing can be provided. The specific physical and functional features of the catheter coupling 400 are not essential to the present disclosure. A catheter coupling suitable in principle for the catheter set is available from the applicant under the registered trademark PERIFIX®.

The fluid admission line 500 has a hose 501 and at least one fluid connector 502. In the present case, a further fluid connector 505 is present. The two fluid connectors 502, 505 are also designated below as proximal fluid connector 505 and distal fluid connector 502. The proximal fluid connector 505 is joined to a proximal end 503 of the hose 501. The distal fluid connector 502 is joined to a distal end 504 of the hose 501. The hose 501 extends between the proximal end 503 and the distal end 504. The fluid admission line 500 has a lumen 506. The lumen 506 is continuous from proximal end to distal end. The distal fluid connector 502 is configured for releasable connection to the capillary attachment 102. The distal fluid connector 502 and the capillary attachment 102 are accordingly adapted to each other in dimensions. The releasable connection is different in different embodiments of the catheter set 1. For example, a plug, clamp and/or screw connection can be provided between the capillary attachment 102 and the distal fluid connector 502. Accordingly, the distal fluid connector 502 and the capillary attachment 102 are designed at least partially complementing each other. The fluid admission line 500 can also be designated as an extension line.

The fixing component 600 is needed for forming the third catheter arrangement 30 (FIG. 6). The fixing component 600 is fastenable to the capillary attachment 102 in a manner that will be described in detail. The fixing component 600 serves for axially fixing the catheter hose 300 inserted into the capillary tube 101 (see FIG. 6). The fixing component 600 is configured accordingly and for this purpose interacts with the capillary 100, specifically the capillary attachment 102, and also with the catheter hose 300.

In the embodiment shown, the catheter set 1 has a plurality of sensors 106, 206, 306 configured for ultrasound-based locating. The sensors 106, 206, 306 can also be designated as first sensor 106, second sensor 206 and third sensor 306. The first sensor 106 is arranged at the distal end of the capillary tube 101. In the present case, the first sensor 106 is embedded and/or integrated in the capillary tube 101. The second sensor 206 is arranged at the distal end 204 of the cannula tube 201. In the present case, the second sensor 206 is embedded and/or integrated in a wall (not shown in detail) of the cannula tube 201. The third sensor 306 is arranged at the distal end 302 of the catheter hose 300. In the present case, the third sensor 306 is embedded and/or integrated in a hose wall (not shown in detail) of the catheter hose 300. The sensors 106, 206, 306 permit ultrasound-based navigating and/or locating. In this way, the relevant component of the catheter set 1 can be positioned with particular precision when placed on the patient. Alternatively or in addition, the position can be monitored by ultrasound after the component has been fitted in place. Sensors for ultrasound-based locating are known in principle to a person skilled in the art. Further specific physical and/or functional features of the sensors 106, 206, 306 are not essential to the present disclosure. Therefore, further explanations concerning the sensors 106, 206, 306 are not needed.

FIGS. 4, 5 and 6 show the different catheter arrangements 10, 20, 30. Specifically:

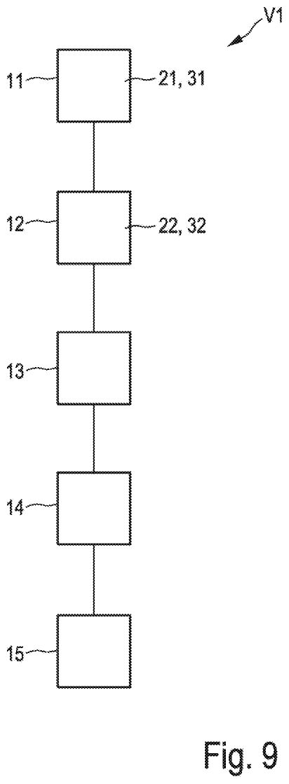

The first catheter arrangement 10 has the catheter hose 300 and the catheter coupling 400. In this first catheter placement, the anaesthetic is administered via the catheter hose 300. For this purpose, the anaesthetic is dispensed by means of a fluid-guiding component attachable to the catheter coupling 400, for example a hose or a syringe, through the catheter coupling 400 into the lumen 303 and from there via the distal end 302 to the nerve that is to be blocked. According to the method shown schematically in FIG. 9, the catheter arrangement 10 can be formed using the catheter set 1. The method illustrated schematically in FIG. 9 is also designated below as first method V1. The first method V1 has the following steps: In a first step 11, the catheter set 1 is provided. In a second step 12, the cannula 200, starting from the state shown in FIG. 1 joined together with the capillary 100, is removed. For this purpose, the cannula attachment 202 is released from the capillary attachment 102. Moreover, the cannula tube 201 is withdrawn in the proximal direction from the capillary tube 101. In a third step 13, the catheter hose 300 is fitted. For this purpose, the distal end 302 of the catheter hose 300 is introduced through the capillary attachment 102 into the capillary tube 101, more precisely the lumen 105, and advanced in the proximal direction. In a fourth step 14, the capillary 100 is removed. For this purpose, the capillary tube 101 together with the capillary attachment 102 is pulled in the proximal direction from the catheter hose 300. In a fifth step 15, the catheter coupling 400 is fastened to the proximal end 301 of the catheter hose 300. Before the catheter coupling 400 is fastened in the present case, the mandrel 305 is pulled in the proximal direction out of the catheter hose 300.

The second catheter arrangement 20 (see FIG. 5) has the capillary 100 and the fluid admission line 500. The (distal) fluid connector 502 is fastened to the catheter attachment 102. The administration of the anaesthetic takes place here via the capillary 100. The anaesthetic is fed through the fluid admission line 500 to the capillary 100. The anaesthetic passes through the lumen 506 of the hose 501 into the lumen 105 of the capillary tube 101 and from there via the distal end 104 to the nerve that is to be blocked.

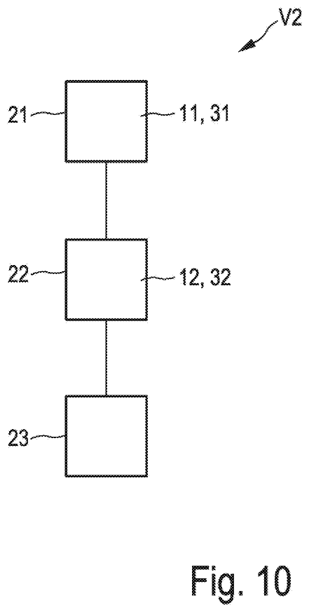

FIG. 10 shows schematically a second method V2 for forming the second catheter arrangement 20 using the catheter set 1. The second method V2 has a first step 21 and a second step 22. The first step 21 and the second step 22 are each identical to the associated step of the first method V1. To avoid repetition, reference is expressly made to what has been said concerning the first method V1. The second method V2 has a third step 23. In this third step, the fluid admission line 500 is fitted in place. For this purpose, the distal fluid connector 502 is fastened to the capillary attachment 102.

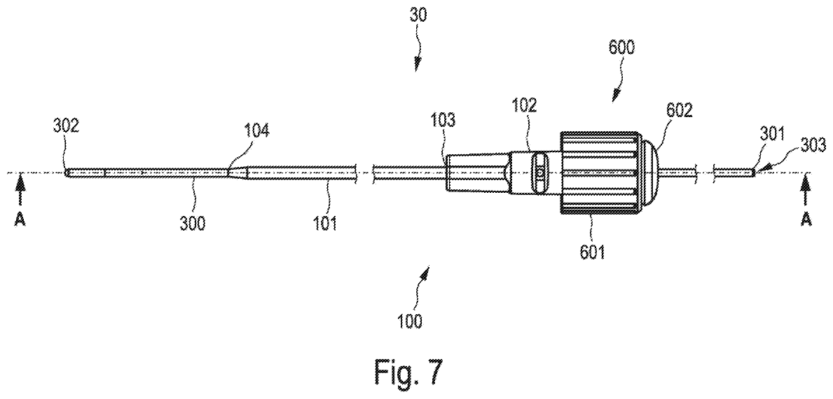

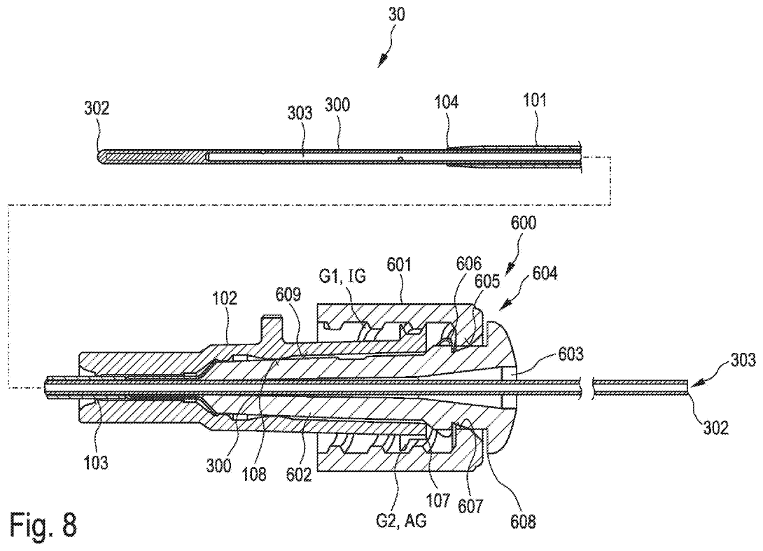

The third catheter arrangement 30 (see in particular FIG. 6) has the capillary 100, the catheter hose 300 and the fixing component 600. Moreover, the third catheter arrangement has the catheter coupling 400. The catheter hose 300 is inserted through the capillary attachment 102 into the capillary tube 101. The distal end 302 of the catheter hose 300 protrudes from the distal end 104 of the capillary tube 101. In the configuration shown in FIG. 6, the catheter hose 300 is fixed, by means of the fixing component 600, in terms of its axial mobility relative to the capillary 100. As regards the specific design and the further function of the fixing component, reference is made to FIGS. 7 and 8.

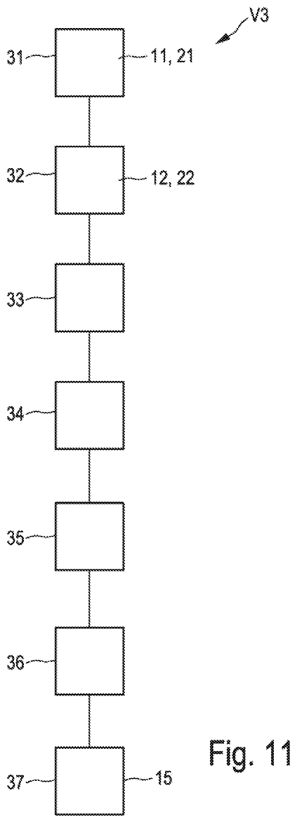

Firstly, referring to FIG. 11, a third method V3 is discussed for forming the third catheter arrangement 30 using the catheter set 1. The third method V3 has a first step 31 and a second step 32. The two steps 31, 32 are identical to the respective step of the first method V1 and of the second method V2. As regards the first step 31, and in order to avoid repetition, reference is expressly made to the explanations concerning the first step 11 of the first method V1. This also applies, mutatis mutandis, to the second step 32. The third method V3 has a third step 33. In the third step 33, the fixing component 600 is mounted on the capillary 100. For this purpose, the fixing component 600 is fastened to the capillary attachment 102. This is done in a manner that will be described in detail. In a fourth step 34, the catheter hose 300 is fitted in place. For this purpose, its distal end 302 is inserted through the fixing component 600 and the capillary attachment 102 into the capillary tube 101 and is advanced distally along the capillary tube 101. In a fifth step 35, the catheter hose 300 is positioned relative to the capillary 100 and therefore also to the fixing component 600. For this purpose, the distal end 302 of the catheter hose 300 is advanced and/or retracted as far as a desired position with respect to the distal end 104 of the capillary tube 101. In a sixth step 36, the catheter hose 300 is fixed. For this purpose, the fixing component 600 is actuated (in a manner to be described in detail) and the catheter hose 300 is axially fixed relative to the capillary 100 under the action of the fixing component. In a seventh step 37 in the present case, the catheter coupling 400 is mounted on the proximal end 301 of the catheter hose 300. The step 37 corresponds to the step 15 of the method V1.

FIGS. 7 and 8 show an alternative and more detailed view of the third catheter arrangement 30. These figures reveal further features of the fixing component 600. The catheter coupling 400 is not shown in FIGS. 7 and 8.

In the embodiment shown, the fixing component 600 has an actuation element 601 and a clamping element 602.

The actuation element 601 is mounted on the capillary attachment 102 in such a way as to be movable relative to the capillary attachment 102 and is operatively connected to the clamping element 602 in a force-transmitting and/or movement-transmitting manner.

The clamping element 602 is received in a receiving recess 107 (see also FIG. 2) of the capillary attachment 102. The clamping element 602 has a lumen 603 oriented coaxially to the capillary tube 101. The lumen 603 is continuous in proximodistal direction between a proximal end and a distal end of the clamping element 602. Said ends of the clamping element 602 are without reference signs. The lumen 603 is configured to receive the catheter hose 300. In the assembly of the components as shown in FIGS. 6 to 8, the catheter hose 300 extends through the lumen 603. The clamping element 602 is dimensionally conformable. For this purpose, the clamping element 602 in the present case is produced from an elastomeric plastic material.

By means of a movement of the actuation element 601, the clamping element 602 is elastically deformable between a clamping configuration (FIG. 8) and an enabling configuration. The enabling configuration is not shown separately in the figures. In the clamping configuration, the clamping element 602 is elastically deformed, on the one hand by the action of the actuation element 601 and on the other hand by the action of the capillary attachment 102, in such a way that the lumen 603 is radially narrowed and, consequently, the axial mobility of the catheter hose 300 is fixed by frictional engagement. In the enabling configuration, the elastic deformation is less pronounced or is absent. The frictional engagement is then cancelled to the extent that the axial mobility of the catheter hose 300 in the lumen 603 is enabled.

In the embodiment shown, the actuation element 601 is movable by screwing relative to the capillary attachment 102. The actuation element 601 has a threaded portion G1. The capillary attachment 102 has a threaded portion G2 complementing the threaded portion G1. In the embodiment shown, the threaded portion G1 is an inner thread IG. The complementary threaded portion G2 is an outer thread AG. The outer thread AG is formed on a proximal end 107 of the capillary attachment 102. The two threaded portions G1, G2 are oriented coaxially with respect to the lumen 603. In the case of a clockwise screwing movement, the actuation element 601 moves distally relative to the capillary attachment 102. An anticlockwise screwing movement causes it to move proximally.

The force-transmitting and/or movement-transmitting operative connection between the actuation element 601 and the clamping element 602 is effected by axial form-fit engagement in the present case. In the circumferential direction, the actuation element 601 and the clamping element 602 interact in a sliding movement. For this purpose, the clamping element 602 has a proximally arranged bearing portion 604. The bearing portion 604 has a slide surface 605. The slide surface 605 is delimited distally by a run-on shoulder 606. The actuation element 601 has a bore (not shown in detail). An inner circumferential surface 607 of the bore is mounted on the slide surface 605 so as to be slidable in the circumferential direction. The edge of the bore runs distally onto the run-on shoulder 606. In the embodiment shown, the bearing portion 604 has a further run-on shoulder 608. The further run-on shoulder 608 can also be designated as proximal run-on shoulder. In a proximal movement of the actuation element 601, the proximal run-on shoulder 608 interacts by form-fit engagement with the edge of the bore.

The receiving recess 107 has an inner cone surface 108. The clamping element 602 has an at least in part complementary outer cone surface 609.

In the clamping configuration, the clamping element 602 is pressed distally into the receiving recess 107. In this way, the two cone surfaces 108, 609 are placed against each other. This leads to said elastic deformation of the clamping element 602 and the resulting radial narrowing of the lumen 603.

It will be understood that that specific physical and functional features of the fixing component 600 that have been explained with reference to FIGS. 7 and 8 are not essential in terms of the present disclosure. However, these features are to be considered advantageous.

1. A catheter set for forming different catheter arrangements for peripheral regional anaesthesia, the catheter set comprising:
a capillary with a capillary tube and a capillary attachment that is joined to a proximal end of the capillary tube;
a cannula with a cannula tube that is inserted or insertable into the capillary tube, and with a cannula attachment that is joined to a proximal end of the cannula tube and is releasably connected or connectable to the capillary attachment;
a catheter hose that is advanceable in a distal direction through the capillary tube;
a catheter coupling configured for fastening to a proximal end of the catheter hose; and
a fluid admission line with a hose and at least one fluid connector that is joined to a distal end of the hose and releasably connectable to the capillary attachment,
the catheter set being configured for selectively forming at least a first catheter arrangement and a second catheter arrangement,
the first catheter arrangement having the catheter hose and the catheter coupling fastened to the proximal end of the catheter hose, the first catheter arrangement configured for dispensing an anaesthetic via the catheter hose, and
the second catheter arrangement having the capillary and the fluid admission line connected to the capillary attachment by the fluid connector, the second catheter arrangement configured for dispensing the anaesthetic via the capillary tube.

2. The catheter set according to claim 1, further comprising a fixing component that is fastenable to the capillary attachment and configured for axially fixing the catheter hose, insertable into the capillary tube, on the capillary,
the catheter set being configured for selectively forming a third catheter arrangement which has the capillary, the catheter hose, the fixing component and the catheter coupling fastened to the proximal end of the catheter hose,
the fixing component being fastened to the capillary attachment, and
the catheter hose being advanced in the distal direction through the capillary tube for dispensing the anaesthetic and axially fixed relative to the capillary by the fixing component.

3. The catheter set according to claim 1, wherein the capillary tube is flexible.

4. The catheter set according to claim 3, wherein the capillary tube is produced from polyurethane.

5. The catheter set according to claim 1, wherein the catheter hose has an extractable mandrel.

6. The catheter set according to claim 1, further comprising a plurality of sensors configured for ultrasound-based locating and mounted on different components of the catheter set.

7. The catheter set according to claim 1, further comprising at least one of:
a first sensor integrated in a distal end of the capillary tube;
a second sensor integrated in a distal end of the cannula tube; and
a third sensor integrated in a distal end of the catheter hose.

8. The catheter set according to claim 2, wherein:
the fixing component has an actuation element and a clamping element that is pliable,
the clamping element is receivable or received in a receiving recess of the capillary attachment and has, in a received state, a lumen that is oriented coaxially to the capillary tube and into which the catheter hose is distally insertable,
the actuation element is mountable or mounted on the capillary attachment in such a way as to be movable relative to the capillary attachment and is operatively connected to the clamping element in a force-transmitting and/or movement-transmitting manner, and
the clamping element, by a movement of the actuation element, is elastically deformable between a clamping configuration, in which the lumen is radially narrowed and an axial mobility of the catheter hose is fixed by frictional engagement, and an enabling configuration, in which the frictional engagement is cancelled and the axial mobility of the catheter hose is enabled.

9. A method for forming a first catheter arrangement with the catheter set according to claim 1, the method comprising the steps of:
removing the cannula, wherein the cannula attachment is released from the capillary attachment, and the cannula tube advanced in the capillary tube is extracted in a proximal direction from the capillary tube;
fitting the catheter hose, wherein the distal end of the catheter hose is inserted into the capillary attachment and advanced in the distal direction along the capillary tube;
removing the capillary, wherein the capillary tube is withdrawn in a proximal direction from the catheter hose; and
fastening the catheter coupling to the proximal end of the catheter hose.

10. A method for forming a second catheter arrangement with the catheter set according to claim 1, the method comprising the steps of:
removing the cannula, wherein the cannula attachment is released from the capillary attachment, and the cannula tube advanced in the capillary tube is extracted in a proximal direction from the capillary tube; and
fitting the fluid admission line, wherein the fluid connector of the latter is connected to the capillary attachment.

11. A method for forming a third catheter arrangement with the catheter set according to claim 2, the method comprising the steps of:
removing the cannula, wherein the cannula attachment is released from the capillary attachment, and the cannula tube advanced in the capillary tube is extracted in a proximal direction from the capillary tube;
fitting the fixing component to the capillary, wherein the fixing component is fastened to the capillary attachment;
fitting the catheter hose, wherein the distal end of the latter is inserted into the capillary attachment and advanced in the distal direction along the capillary tube;
positioning the catheter hose, wherein the distal end of the latter is advanced as far as a desired position with respect to the distal end of the capillary tube;
fixing the catheter hose, wherein the fixing component is actuated and the catheter hose is axially fixed relative to the capillary under the action of the fixing component; and
fastening the catheter coupling to the proximal end of the catheter hose.
